(19) United States
(12) Patent Application Publication
(10) Pub. No.: US 2022/0287594 A1
(43) Pub. Date: Sep. 15, 2022

(54) PHYSICAL ACTIVITY MONITORING DEVICE

(71) Applicant: Murata Manufacturing Co., Ltd., Nagaokakyo-shi (JP)

(72) Inventors: Yutaka TAKAMARU, Nagaokakyo-shi (JP); Atsushi NAITO, Nagaokakyo-shi (JP); Naoki KAWARA, Nagaokakyo-shi (JP)

(21) Appl. No.: 17/830,720

(22) Filed: Jun. 2, 2022

Related U.S. Application Data
(63) Continuation of application No. PCT/JP2020/045557, filed on Dec. 8, 2020.

(30) Foreign Application Priority Data
Dec. 12, 2019 (JP) 2019-224142

(51) Int. Cl. A61B 5/11; A61B 5/00

(52) U.S. Cl. CPC A61B 5/1118; A61B 5/6829; A61B 5/1116; A61B 5/1114; A61B 5/1101

(57) ABSTRACT

A physical activity monitoring device is provided that includes a muscle activity sensor, an acceleration sensor, and a computation unit. The acceleration sensor can be attached to a leg and can output a first monitoring signal corresponding to an activity of the leg. The muscle activity sensor can be attached to the leg and can output a second monitoring signal corresponding to an activity of a muscle and/or a tendon of the leg. The computation unit can detect the load condition of the body of a wearer or user that includes the body position of the wearer or user by using the first monitoring signal and the second monitoring signal.

### CROSS REFERENCE TO RELATED APPLICATIONS

This application is a continuation of PCT Application No. PCT/JP2020/045557, filed Dec. 8, 2020, which claims priority to Japanese Patent Application No. 2019-224142, filed Dec. 12, 2019, the entire contents of each of which are hereby incorporated in their entirety.

### TECHNICAL FIELD

The present invention relates to a system and method configured for monitoring physical activities including muscle activities.

### BACKGROUND

Japanese Unexamined Patent Application Publication No. 2016-150179 (hereinafter “Patent Document 1”) discloses a motion measurement device for measuring the motion of an ankle. The motion measurement device described in Patent Document 1 includes an acceleration sensor configured to be attached to an ankle.

In Patent Document 1, the acceleration sensor outputs a signal corresponding to the motion of the ankle. The motion measurement device uses the output signal from the acceleration sensor to measure the motion of the ankle.

The known motion measurement devices, such as the motion measurement device disclosed in Patent Document 1, however, cannot measure all kinds of physical activities. For example, multiple kinds of body positions cannot be measured by the known motion measurement devices.

### SUMMARY OF THE INVENTION

Accordingly, it is an object of the present invention to provide a physical activity monitoring device configured to monitor an increased variety of physical activities.

Thus, a physical activity monitoring device according to an exemplary aspect includes an acceleration sensor, a muscle activity sensor, and a computation unit. The acceleration sensor is configured to be attached to a leg and to output a first monitoring signal corresponding to an activity of the leg. The muscle activity sensor is configured to be attached to the leg and to output a second monitoring signal corresponding to an activity of a muscle and/or a tendon of the leg. The computation unit is configured to detect the load condition of the body of the wearer including the body position of the wearer by using the first monitoring signal and the second monitoring signal.

In this configuration of the exemplary aspect, the first monitoring signal is used to monitor the orientation (e.g., position) of the leg, and the second monitoring signal is used to monitor the load condition of the leg. Here, particular body positions of a wearer strongly correlate with particular combinations of the orientation (e.g., position) of a leg and the load condition of the leg. For this reason, it is possible to monitor the body position of the wearer by combining the first monitoring signal and the second monitoring signal.

The exemplary aspects described herein enable monitoring various physical activities including differentiation of multiple body positions.

### BRIEF DESCRIPTION OF DRAWINGS

FIG. 1 is a functional block diagram illustrating a configuration of a physical activity monitoring device according to a first exemplary embodiment.

FIG. 2(A) is a side view illustrating a manner of attachment of the physical activity monitoring device to a monitoring subject; FIG. 2(B) is a top view illustrating the manner of attachment.

FIG. 3(A) is a simplified illustration depicting a standing position as a body position; FIG. 3(B) is a simplified illustration depicting a sitting position (chair sitting position) as a body position.

FIG. 4(A) is a simplified illustration depicting a lying position (supine position) as a body position; FIG. 4(B) is a simplified illustration depicting a lying position (prone position) as a body position; FIG. 4(C) is a simplified illustration depicting a lying position (left lateral recumbent position) as a body position; FIG. 4(D) is a simplified illustration depicting a lying position (right lateral recumbent position) as a body position.

FIGS. 5(A), 5(B), and 5(C) are graphs illustrating examples of waveform of a second monitoring signal.

FIG. 6 is a graph illustrating an example of values of muscle activity index of different body positions.

FIG. 7 provides graphs illustrating an example of values of acceleration index of different body positions.

FIG. 8 is a first table indicating the relationship between the muscle activity index and the acceleration index, and the body position.

FIG. 9 is a graph illustrating the relationship between the value of the muscle activity index and the value of the acceleration index, and the body position.

FIG. 10 is a flowchart illustrating a first example of a physical activity monitoring method according to the first exemplary embodiment.

FIG. 11 is a second table indicating the relationship between the muscle activity index and the acceleration index, and the body position.

FIG. 12 is a flowchart illustrating a second example of the physical activity monitoring method according to the first exemplary embodiment.

FIGS. 13(A), 13(B), 13(C), 13(D), and 13(E) are third tables indicating the relationship between the muscle activity index and the acceleration index, and the body position.

FIG. 14 is a flowchart illustrating a third example of the physical activity monitoring method according to the first exemplary embodiment.

FIG. 15 is a simplified illustration depicting a manner of attachment of a physical activity monitoring device according to a second exemplary embodiment.

FIG. 16 is a simplified illustration depicting a cross-legged sitting position in the state in which the physical activity monitoring device according to the second exemplary embodiment is attached.

FIG. 17 provides graphs illustrating an example of values of acceleration index of the lying position (left lateral recumbent position), the lying position (right lateral recumbent position), and a sitting position (cross-legged sitting position).

FIG. 18 is a fourth table indicating the relationship between the muscle activity index and the acceleration index, and the body position.

FIG. 19 provides a flowchart illustrating a first example of the physical activity monitoring method according to the second exemplary embodiment.

FIG. 20 provides a flowchart illustrating the first example of the physical activity monitoring method according to the second exemplary embodiment.

FIG. 21 is a functional block diagram of a physical activity monitoring device according to an additional exemplary embodiment.

### DETAILED DESCRIPTION OF EMBODIMENTS

(First Exemplary Embodiment)

A physical activity monitoring device according to a first exemplary embodiment will be described with reference to the drawings.

(Outline of Functional Configuration)

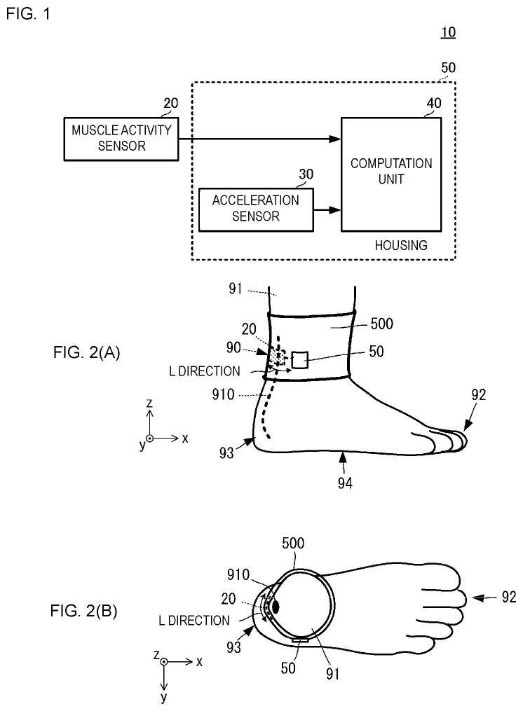

FIG. 1 is a functional block diagram illustrating a configuration of the physical activity monitoring device according to the first exemplary embodiment. As illustrated in FIG. 1, a physical activity monitoring device 10 includes a muscle activity sensor 20, an acceleration sensor 30, and a computation unit 40. The muscle activity sensor 20 and the acceleration sensor 30 are connected to the computation unit 40. For example, as illustrated in FIG. 1, the acceleration sensor 30 and the computation unit 40 are housed in a housing 50. The muscle activity sensor 20 and the housing 50 are attached to a monitoring target part of a person (e.g. a wearer or user) who is targeted for physical activity monitoring (refer to FIGS. 2(A) and 2(B) described later).

The muscle activity sensor 20 includes a piezoelectric sensor formed by, for example, a flat piezoelectric film. The muscle activity sensor 20 is configured to generate a second monitoring signal of the waveform and level corresponding to an activity of muscles or tendons of the monitoring target part of the wearer. The muscle activity sensor 20 is also configured to output the second monitoring signal to the computation unit 40. In an additional aspect, the muscle activity sensor 20 can be a sensor configured to detect muscle activities in accordance with another method, such as an electromyography sensor (electromyograph), for example.

The acceleration sensor 30 can be configured to monitor three kinds of accelerations (ax, ay, az) along three perpendicular axes (x axis, y axis, z axis). Thus, the acceleration sensor 30 is configured to generate a first monitoring signal representing information including the x-axis acceleration ax, the y-axis acceleration ay, and the z-axis acceleration az of the three kinds of accelerations of the three perpendicular axes. The acceleration sensor 30 is also configured to output the first monitoring signal to the computation unit 40.

The computation unit 40 is implemented by, for example, a program for performing an operation of detecting the load condition of a body including, for example, a body position, which will be described later, a storage medium storing the program, and an operational element for running this program, such as a central processing unit (CPU), for example. In an exemplary aspect, the computation unit 40 may be, for example, a microcomputer configured to implement a method of monitoring physical activities including the body position described later.

In operation, the computation unit 40 detects the load condition of the body of the wearer including the body position of the wearer by using the first monitoring signal and the second monitoring signal. As will be more specifically described later, the computation unit 40 detects a standing position, a sitting position, or lying position as the body position of the wearer. The sitting position includes, for example, a chair sitting position (e.g., sitting on a chair). The lying position includes, for example, a prone position, a supine position, a left lateral recumbent position, and a right lateral recumbent position.

(Manner of Attachment to Monitoring Subject)

FIG. 2(A) is a side view illustrating a manner of attachment of the physical activity monitoring device to a monitoring subject. FIG. 2(B) is a top view illustrating the manner of attachment. As illustrated in FIGS. 2(A) and 2(B), the housing 50 housing the acceleration sensor 30 and the computation unit 40, and the muscle activity sensor 20 are fixed to a body supporter 500. In an exemplary aspect, the muscle activity sensor 20 may be integrated with the body supporter 500.

The body supporter 500 is shaped as a tube. The body supporter 500 is made of a stretchable material, so that the body supporter 500 changes its shape with the motion of the body. Preferably, the body supporter 500 can be made of a material that does not prevent displacement of the muscle activity sensor 20 as much as possible. For example, a cotton/acrylic blend, a polyester/cotton blend, a cotton/linen blend, an acrylic/wool blend, a wool/nylon blend, a material mixed with animal hair, silk, spun-silk yarn, or noil silk yarn may be used in various exemplary aspects. The body supporter 500 is attached to an ankle 91 to cover the ankle 91. The body supporter 500 may be configured to cover a portion other than the ankle 91. The body supporter 500 may be configured in the form of, for example, a sock.

The muscle activity sensor 20 is positioned over, for example, an Achilles tendon 910 as illustrated in FIGS. 2(A) and 2(B). In particular, the muscle activity sensor 20 is configured to be positioned around an ankle circumference 90 in an exemplary aspect. As a result, the muscle activity sensor 20 can monitor activities of tendons and/or muscles of and near the ankle 91 with high sensitivity and output the second monitoring signal according to the monitoring result. This configuration enables the signal level and waveform of the second monitoring signal that represent activities of tendons and/or muscles of and near the ankle 91 to have a high sensitivity.

The acceleration sensor 30 is positioned on the outside of the ankle 91.

The acceleration sensor 30 detects an acceleration parallel to the direction connecting a toe tip 92 and a heel 93 and outputs the acceleration as the x-axis acceleration ax. To detect the x-axis acceleration ax, the direction from the heel 93 to the toe tip 92 is determined as a plus direction, and the direction from the toe tip 92 to the heel 93 as a minus direction according to the exemplary aspect.

Moreover, the acceleration sensor 30 detects an acceleration parallel to the direction perpendicular to a side of the ankle 91 and outputs the acceleration as the y-axis acceleration ay. To detect the y-axis acceleration ay, the direction from the ankle 91 to the outside is determined as a plus direction, and the direction from the ankle 91 to the inside as a minus direction according to the exemplary aspect.

Furthermore, the acceleration sensor 30 detects an acceleration parallel to the direction in which the ankle 91 is stretchable, that is, the direction from a sole 94 to the ankle 91 and outputs the acceleration as the z-axis acceleration az. To detect the z-axis acceleration az, the direction from the sole 94 to the ankle 91 is determined as a plus direction, and the direction from the ankle 91 to the sole 94 as a minus direction according to the exemplary aspect.

(Description of Body Position)

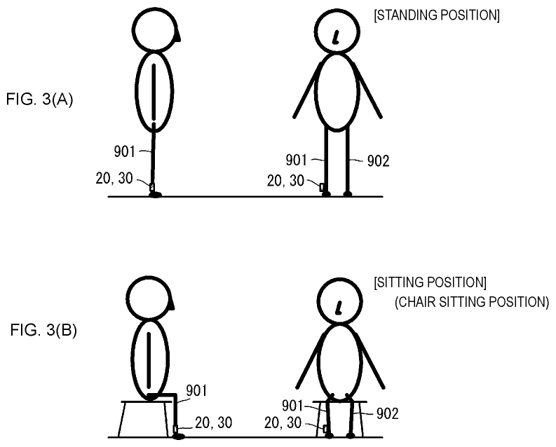

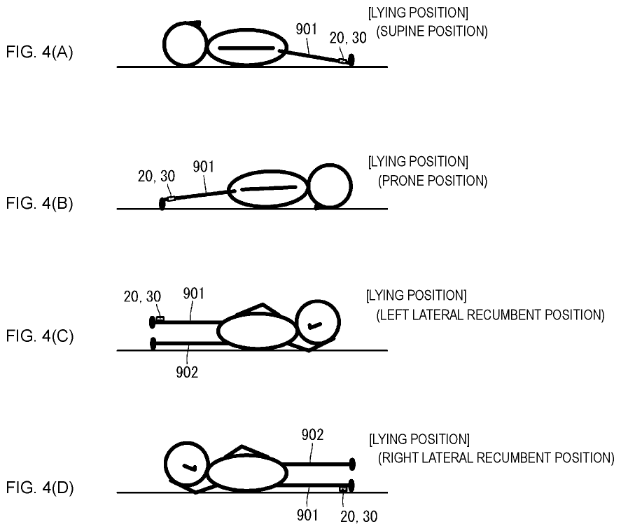

With the configuration described above, the physical activity monitoring device 10 is configured to detect muscle activities of and near the ankle 91 and the body positions described below. FIG. 3(A) is a simplified illustration depicting a standing position as a body position. FIG. 3(B) is a simplified illustration depicting a sitting position (e.g., a chair sitting position) as a body position. FIG. 4(A) is a simplified illustration depicting a lying position (e.g., a supine position) as a body position. FIG. 4(B) is a simplified illustration depicting a lying position (e.g., a prone position) as a body position. FIG. 4(C) is a simplified illustration depicting a lying position (e.g., a left lateral recumbent position) as a body position. FIG. 4(D) is a simplified illustration depicting a lying position (e.g., a right lateral recumbent position) as a body position.

As illustrated in FIGS. 3(A), 3(B), 4(A), 4(B), 4(C), and 4(D), the muscle activity sensor 20 and the acceleration sensor 30 are attached to the ankle 91 (refer to FIGS. 2(A) and 2(B)) of a right leg 901. The axial directions of acceleration are set as described above.

In the standing position illustrated in FIG. 3(A), the direction of gravity is the minus direction of z-axis acceleration. The x-axis acceleration and y-axis acceleration are almost zero. To maintain the standing position, a relatively high level of muscle activity occurs at the ankle 91.

In the sitting position (e.g., the chair sitting position) illustrated in the FIG. 3(B), the direction of gravity is the minus direction of z-axis acceleration. The x-axis acceleration and y-axis acceleration are almost zero. It should be appreciated that the sitting position does not cause a relatively high level of muscle activity at the ankle 91.

In the lying position (e.g., a supine position) illustrated in FIG. 4(A), the direction of gravity is the minus direction of x-axis acceleration. The y-axis acceleration and z-axis acceleration are almost zero. Similar to the sitting position, the lying position does not cause a relatively high level of muscle activity at the ankle 91.

In the lying position (e.g., a prone position) illustrated in FIG. 4(B), the direction of gravity is the plus direction of x-axis acceleration. The y-axis acceleration and z-axis acceleration are almost zero. The lying position does not cause a relatively high level of muscle activity at the ankle 91.

In the lying position (e.g., a left lateral recumbent position) illustrated in FIG. 4(C), a left leg 902 is under the right leg 901, and the direction of gravity is the minus direction of the y-axis acceleration ay. The x-axis acceleration and z-axis acceleration are almost zero. The lying position does not cause a relatively high level of muscle activity at the ankle 91.

In the lying position (e.g., a right lateral recumbent position) illustrated in FIG. 4(D), the right leg 901 is under the left leg 902, and the direction of gravity is the plus direction of the y-axis acceleration ay. The x-axis acceleration and z-axis acceleration are almost zero. The lying position does not cause a relatively high level of muscle activity at the ankle 91.

As described above, the combination of the level of muscle activity, the level of x-axis acceleration, the level of y-axis acceleration, and the level of z-axis acceleration varies with the body position of the wearer. Thus, the physical activity monitoring device 10 is configured to detect differences in the combination to detect different body positions.

(Muscle Activity index)

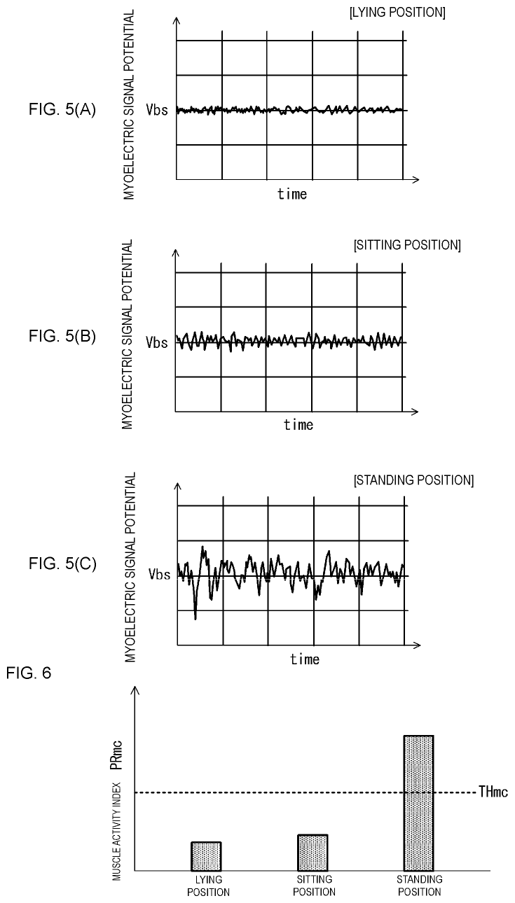

The computation unit 40 is configured to calculate a muscle activity index PRmc by using the second monitoring signal from the muscle activity sensor 20. FIGS. 5(A), 5(B), and 5(C) are graphs illustrating examples of waveform of the second monitoring signal. In FIGS. 5(A), 5(B), and 5(C), the second monitoring signal is a signal of the muscle activity sensor 20 (for example, an output signal from a piezoelectric film), and the level of the second monitoring signal is the potential of the signal of the muscle activity sensor 20. FIG. 5(A) indicates the case of the lying position; FIG. 5(B) indicates the case of the sitting position; FIG. 5(C) indicates the case of the standing position.

As illustrated in FIGS. 5(A) and 5(B), in the lying position and the sitting position, the load on muscles and tendons of and near the ankle 91 is relatively small. The level (potential) of the second monitoring signal (signal of the muscle activity sensor 20) thus fluctuates mildly in the range of values close to a reference value (Vbs). By contrast, as illustrated in FIG. 5(C), in the standing position, the load on muscles and tendons of and near the ankle 91 is relatively large. The level (potential) of the second monitoring signal (signal of the muscle activity sensor 20) thus fluctuates greatly in the range including values far from the reference value (Vbs).

The computation unit 40 is configured to calculate the muscle activity index PRmc by using the difference between an instantaneous value of the level (potential) of the second monitoring signal (signal of the muscle activity sensor 20) and the reference value (Vbs). More specifically, the computation unit 40 can be configured to calculate the muscle activity index PRmc by firstly calculating a time integral of the absolute value of the difference between an instantaneous value of the level (potential) of the second monitoring signal (signal of the muscle activity sensor 20) and the reference value (Vbs) and secondly dividing the time integral by the number of samples (integration time). As a result, the computation unit 40 calculates, as the muscle activity index PRmc, the time average of fluctuations in the level of the second monitoring signal.

According to this calculation process, the muscle activity index PRmc can be values indicated in FIG. 6. FIG. 6 is a graph illustrating an example of values of the muscle activity index of different body positions.

In the lying position and the sitting position, the level of the second monitoring signal fluctuates mildly as illustrated in FIGS. 5(A) and 5(B), and thus, the lying position and the sitting position indicate relatively small values of the muscle activity index PRmc as illustrated in FIG. 6. By contrast, in the standing position, the level of the second monitoring signal fluctuates greatly as illustrated in FIG. 5(C), and thus, the standing position indicates a relatively large value of the muscle activity index PRmc as illustrated in FIG. 6. As such, the muscle activity index PRmc varies between the lying position and the sitting position, and the standing position.

By using this, the computation unit 40 sets a threshold THmc of the muscle activity index PRmc for differentiation. The threshold THmc can be determined by using a suitable value, for example, between the muscle activity index PRmc in the lying position and the muscle activity index PRmc in the sitting position, and the muscle activity index PRmc in the standing position that are calculated in advance.

Accordingly, when the muscle activity index PRmc is equal to or larger than the threshold THmc, the computation unit 40 detects the standing position, whereas when the muscle activity index PRmc is smaller than the threshold THmc, the computation unit 40 detects the lying position or the sitting position.

(Acceleration Index)

The computation unit 40 calculates an acceleration index by using the first monitoring signal from the acceleration sensor 30. For example, the computation unit 40 calculates an acceleration index by firstly calculating a time integral of the level of the first monitoring signal (acceleration detection signal) and secondly dividing the time integral by the number of samples (integration time). As such, the computation unit 40 can be configured to calculate the time average of acceleration as the acceleration index. The computation unit 40 calculates the acceleration index individually for the x axis, the y axis, and the z axis. Concerning acceleration, an instantaneous value can be used as the acceleration index. In the following description, the x-axis acceleration index is ax, the y-axis acceleration index is ay, and the z-axis acceleration index is az.

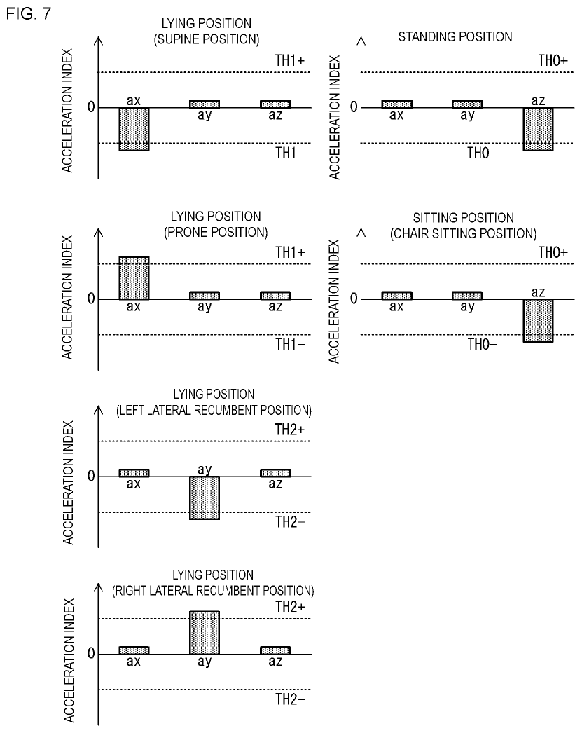

FIG. 7 provides graphs illustrating an example of values of the acceleration index of different body positions. In FIG. 7, the reference value of acceleration (value of no acceleration) is 0 as an example. The following describes cases of the manners of attachment illustrated in FIGS. 3 and 4.

In the lying position (e.g., a supine position), the x-axis acceleration index ax is a relatively large minus value (negative value), and the y-axis acceleration index ay and the z-axis acceleration index az are approximately zero (almost equal to the reference value). In the lying position (e.g., a prone position), the x-axis acceleration index ax is a relatively large plus value (positive value), and the y-axis acceleration index ay and the z-axis acceleration index az are approximately zero (almost equal to the reference value).

In the lying position (e.g., a left lateral recumbent position), the y-axis acceleration index ay is a relatively large minus value (negative value), and the x-axis acceleration index ax and the z-axis acceleration index az are approximately zero (almost equal to the reference value). In the lying position (e.g., a right lateral recumbent position), the y-axis acceleration index ay is a relatively large plus value (positive value), and the x-axis acceleration index ax and the z-axis acceleration index az are approximately zero (almost equal to the reference value).

In the standing position and the sitting position (e.g., a chair sitting position), the z-axis acceleration index az is a relatively large minus value (negative value), and the x-axis acceleration index ax and the y-axis acceleration index ay are approximately zero (almost equal to the reference value).

As described above, the pattern of the x-axis acceleration index ax and the pattern of the y-axis acceleration index ay vary among the lying position (e.g., supine position), the lying position (e.g., prone position), the lying position (e.g., left lateral recumbent position), and the lying position (e.g., right lateral recumbent position). The pattern of the z-axis acceleration index az varies between these kinds of the lying position and the standing position or the sitting position (e.g., chair sitting position).

By using this, the computation unit 40 sets thresholds TH1+, TH1−, TH2+, TH2−, TH0+, and TH0− of the acceleration index for differentiation. The thresholds TH1+, TH1−, TH2+, TH2−, TH0+, and TH0− can be determined by using suitable values, for example, in accordance with the acceleration index obtained in advance with respect to the lying position, the sitting position, and the standing position, similarly to the threshold THmc of the muscle activity index PRmc.

Accordingly, when the x-axis acceleration index ax is equal to or smaller than the threshold TH1−, and the y-axis acceleration index ay and the z-axis acceleration index az are larger than the threshold TH1− and smaller than the threshold TH1+, the computation unit 40 detects the lying position (e.g., supine position). When the x-axis acceleration index ax is equal to or larger than the threshold TH1−, and the y-axis acceleration index ay and the z-axis acceleration index az are larger than the threshold TH1− and smaller than the threshold TH1+, the computation unit 40 detects the lying position (e.g., prone position).

When the y-axis acceleration index ay is equal to or smaller than the threshold TH2−, and the x-axis acceleration index ax and the z-axis acceleration index az are larger than the threshold TH2− and smaller than the threshold TH2+, the computation unit 40 detects the lying position (e.g., left lateral recumbent position). When the y-axis acceleration index ay is equal to or larger than the threshold TH2+, and the x-axis acceleration index ax and the z-axis acceleration index az are larger than the threshold TH2− and smaller than the threshold TH2+, the computation unit 40 detects the lying position (e.g., right lateral recumbent position).

When the z-axis acceleration index az is equal to or smaller than the threshold TH0−, and the x-axis acceleration index ax and the y-axis acceleration index ay are larger than the threshold TH0− and smaller than the threshold TH0+, the computation unit 40 detects the standing position or the sitting position (e.g., chair sitting position).

(Specific Example of Differentiation and Detection of Body Position by Computation Unit 40)

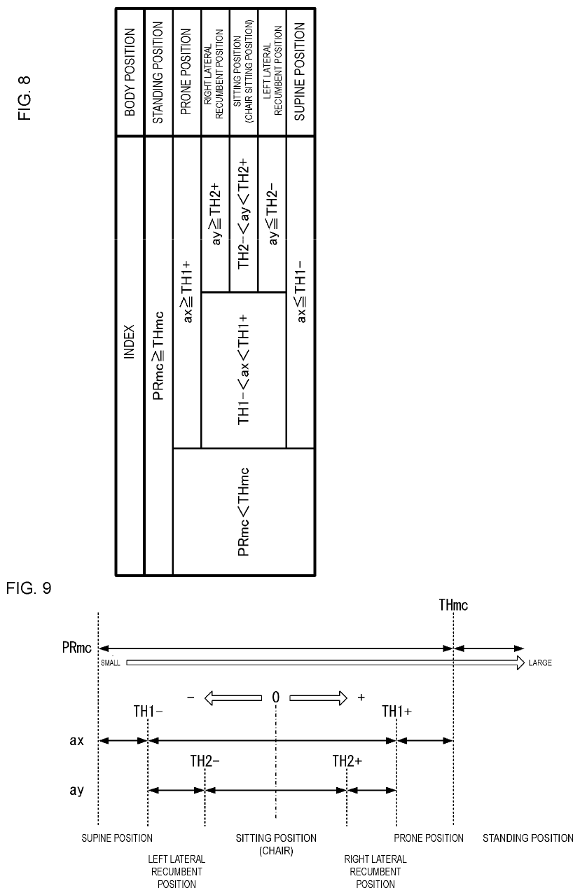

FIG. 8 is a first table indicating the relationship between the muscle activity index and the acceleration index, and the body position. FIG. 9 is a graph illustrating the relationship between the value of the muscle activity index and the value of the acceleration index, and the body position. In the example in FIGS. 8 and 9, the z-axis acceleration index az is not used to detect the body position.

After calculating the muscle activity index PRmc, the x-axis acceleration index ax, and the y-axis acceleration index ay, the computation unit 40 differentiates and detects body positions in accordance with rules indicated in FIGS. 8 and 9 by using these indexes.

Specifically, when the muscle activity index PRmc is equal to or larger than the threshold THmc, the computation unit 40 detects the standing position. When the muscle activity index PRmc is smaller than the threshold THmc, the computation unit 40 detects body positions as described below in accordance with the x-axis acceleration index ax and the y-axis acceleration index ay.

When the x-axis acceleration index ax is equal to or larger than the threshold TH1+, the computation unit 40 detects the lying position (e.g., prone position). In this case, the computation unit 40 can more accurately detect the lying position (e.g., prone position) when the computation unit 40 also determines the y-axis acceleration index ay to be larger than the threshold TH1− and smaller than the threshold TH1+.

When the x-axis acceleration index ax is equal to or smaller than the threshold TH1−, the computation unit 40 detects the lying position (e.g., supine position). In this case, the computation unit 40 can more accurately detect the lying position (e.g., supine position) when the computation unit 40 also determines the y-axis acceleration index ay to be larger than the threshold TH1− and smaller than the threshold TH1+.

When the x-axis acceleration index ax is larger than the threshold TH1− and smaller than the threshold TH1+, the computation unit 40 detects body positions as described below in accordance with the y-axis acceleration index ay.

When the y-axis acceleration index ay is equal to or larger than the threshold TH2+, the computation unit 40 detects the lying position (e.g., right lateral recumbent position). When the y-axis acceleration index ay is equal to or smaller than the threshold TH2−, the computation unit 40 detects the lying position (e.g., left lateral recumbent position). When the y-axis acceleration index ay is larger than the threshold TH2− and smaller than the threshold TH2+, the computation unit 40 detects the sitting position (e.g., chair sitting position).

As described above, with the use of the configurations and operations of the present embodiment, the physical activity monitoring device 10 is configured to detect multiple kinds of body positions, in other words, an increased variety of physical activities.

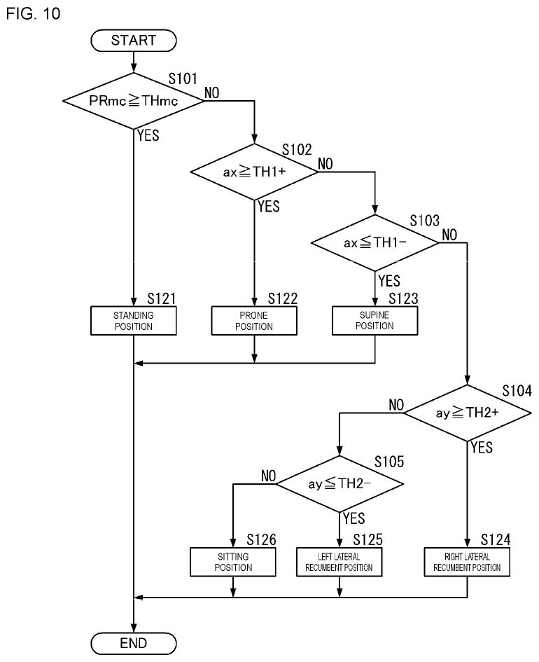

This kind of body position detection can be realized by performing, for example, a process following a flowchart illustrated in FIG. 10. FIG. 10 is a flowchart illustrating a first example of a physical activity monitoring method according to the first embodiment.

When the muscle activity index PRmc is equal to or larger than the threshold THmc (YES in S101), the computation unit 40 detects the standing position (S121). When the muscle activity index PRmc is smaller than the threshold THmc (NO in S101), and the x-axis acceleration index ax is equal to or larger than the threshold TH1+ (YES in S102), the computation unit 40 detects the lying position (e.g., prone position) (S122).

When the x-axis acceleration index ax is smaller than the threshold TH1+ (NO in S102) and equal to or smaller than the threshold TH1− (YES in S103), the computation unit 40 detects the lying position (e.g., supine position) (S123).

When the x-axis acceleration index ax is not equal to or smaller than the threshold TH1− (NO in S103), and the y-axis acceleration index ay is equal to or larger than the threshold TH2+ (YES in S104), the computation unit 40 detects the lying position (e.g., right lateral recumbent position) (S124).

When the y-axis acceleration index ay is smaller than the threshold TH2+ (NO in S104) and equal to or smaller than the threshold TH2− (YES in S105), the computation unit 40 detects the lying position (e.g., left lateral recumbent position) (S125). When the y-axis acceleration index ay is not equal to or smaller than the threshold TH2− (NO in S105), the computation unit 40 detects the sitting position (e.g., chair sitting position) (S126).

(Method of Detecting Body Position With Additional Use of Z-Axis Acceleration az)

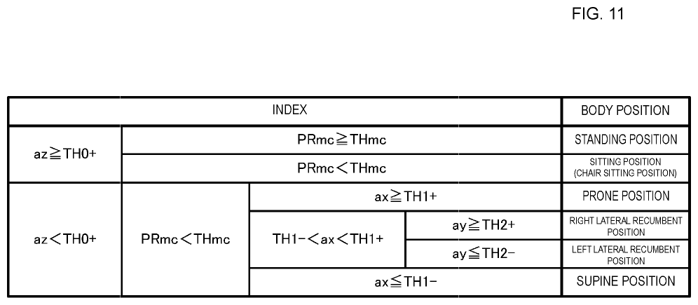

The physical activity monitoring device 10 can also detect a body position by additionally using the z-axis acceleration az. FIG. 11 is a second table indicating the relationship between the muscle activity index and the acceleration index, and the body position. Descriptions of the same details as the case without using the z-axis acceleration az are omitted.

Specifically, when the z-axis acceleration az is equal to or larger than the threshold TH0+, the computation unit 40 detects the standing position or the sitting position (e.g., chair sitting position). When the muscle activity index PRmc is equal to or larger than the threshold THmc, the computation unit 40 detects the standing position. When the muscle activity index PRmc is smaller than the threshold THmc, the computation unit 40 detects the sitting position (e.g., chair sitting position).

When the z-axis acceleration az is smaller than the threshold TH0+, and the muscle activity index PRmc is smaller than the threshold THmc, the computation unit 40 detects body positions in accordance with the following process.

When the x-axis acceleration index ax is equal to or larger than the threshold TH1+, the computation unit 40 detects the lying position (e.g., prone position). When the x-axis acceleration index ax is equal to or smaller than the threshold TH1−, the computation unit 40 detects the lying position (e.g., supine position).

When the x-axis acceleration index ax is larger than the threshold TH1− and smaller than the threshold TH1+, the computation unit 40 detects body positions as described below in accordance with the y-axis acceleration index ay.

When the y-axis acceleration index ay is equal to or larger than the threshold TH2+, the computation unit 40 detects the lying position (e.g., right lateral recumbent position). When the y-axis acceleration index ay is equal to or smaller than the threshold TH2−, the computation unit 40 detects the lying position (e.g., left lateral recumbent position).

As described above, with the use of the z-axis acceleration az, the physical activity monitoring device 10 can be configured to detect multiple kinds of body positions, in other words, an increased variety of physical activities.

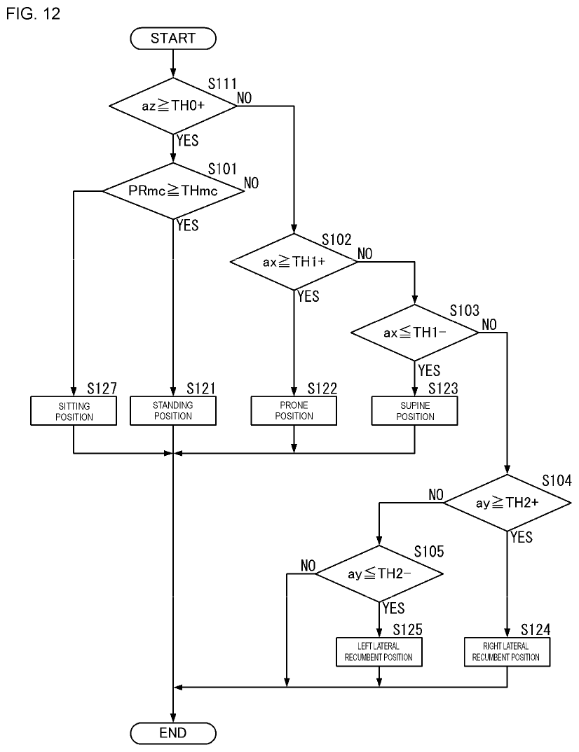

This kind of body position detection can be realized by performing, for example, a process following a flowchart illustrated in FIG. 12. In particular, FIG. 12 is a flowchart illustrating a second example of the physical activity monitoring method according to the first exemplary embodiment.

As shown in FIG. 12, when the z-axis acceleration az is equal to or larger than the threshold TH0+ (YES in S111), and the muscle activity index PRmc is equal to or larger than the threshold THmc (YES in S101), the computation unit 40 detects the standing position (S121). When the z-axis acceleration az is equal to or larger than the threshold TH0+ (YES in S111), and the muscle activity index PRmc is smaller than the threshold THmc (NO in S101), the computation unit 40 detects the sitting position (e.g., chair sitting position) (S127).

When the z-axis acceleration az is smaller than the threshold TH0+ (NO in S111), and the x-axis acceleration index ax is equal to or larger than the threshold TH1+ (YES in S102), the computation unit 40 detects the lying position (e.g., prone position) (S122). When the x-axis acceleration index ax is smaller than the threshold TH1+ (NO in S102) and equal to or smaller than the threshold TH1− (YES in S103), the computation unit 40 detects the lying position (e.g., supine position) (S123).

When the x-axis acceleration index ax is not equal to or smaller than the threshold TH1− (NO in S103), and the y-axis acceleration index ay is equal to or larger than the threshold TH2+ (YES in S104), the computation unit 40 detects the lying position (e.g., right lateral recumbent position) (S124). When the y-axis acceleration index ay is smaller than the threshold TH2+ (NO in S104) and equal to or smaller than the threshold TH2− (YES in S105), the computation unit 40 detects the lying position (e.g., left lateral recumbent position) (S125).

(Method of Detecting Body Position With Use of Absolute Value of Acceleration)

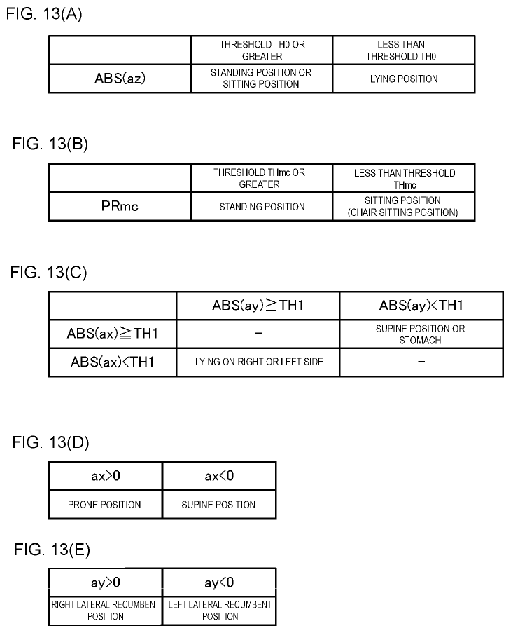

The physical activity monitoring device 10 can also be configured to detect a body position by using the absolute value of acceleration. FIGS. 13(A), 13(B), 13(C), 13(D), and 13(E) are third tables indicating the relationship between the muscle activity index and the acceleration index, and the body position.

Specifically, as illustrated in FIG. 13(A), given that the absolute value of the z-axis acceleration az is a z-axis acceleration index absolute value ABS(az), when the z-axis acceleration index absolute value ABS(az) is equal to or larger than a threshold TH0, the computation unit 40 detects the standing position or the sitting position (e.g., chair sitting position). When the z-axis acceleration index absolute value ABS(az) is smaller than the threshold TH0, the computation unit 40 detects the lying position. The threshold TH0 can be set by using the absolute value of the threshold TH0+ or the threshold TH0− in exemplary aspects.

As illustrated in FIG. 13(B), when the muscle activity index PRmc is equal to or larger than the threshold THmc, the computation unit 40 detects the standing position. Alternatively, when the muscle activity index PRmc is smaller than the threshold THmc, the computation unit 40 detects the sitting position (e.g., chair sitting position).

As illustrated in FIG. 13(C), given that the absolute value of the x-axis acceleration ax is an x-axis acceleration index absolute value ABS(ax), and the absolute value of the y-axis acceleration ay is a y-axis acceleration index absolute value ABS(ay), when the x-axis acceleration index absolute value ABS(ax) is equal to or larger than a threshold TH1, and the y-axis acceleration index absolute value ABS(ay) is smaller than the threshold TH1, the computation unit 40 detects the lying position (e.g., supine position) or the lying position (e.g., prone position). When the x-axis acceleration index absolute value ABS(ax) is smaller than the threshold TH1, and the y-axis acceleration index absolute value ABS(ay) is equal to or larger than the threshold TH1, the computation unit 40 detects the lying position (e.g., right lateral recumbent position) or the lying position (e.g., left lateral recumbent position). The threshold TH1 can be set by using the absolute value of the threshold TH1+ or the threshold TH1−.

As illustrated in FIG. 13(D), after the computation unit 40 performs the detection operation as indicated in FIG. 13(C), when the x-axis acceleration index ax is a plus value (positive value), the computation unit 40 detects the lying position (e.g., prone position); when the x-axis acceleration index ax is a minus value (negative value), the computation unit 40 detects the lying position (e.g., supine position). After the computation unit 40 performs the detection operation as indicated in FIG. 13(C), when the y-axis acceleration index ay is a plus value (positive value), the computation unit 40 detects the lying position (e.g., right lateral recumbent position); when the y-axis acceleration index ay is a minus value (negative value), the computation unit 40 detects the lying position (e.g., left lateral recumbent position).

As described above, with the use of the absolute value of acceleration, the physical activity monitoring device 10 can be configured to also detect multiple kinds of body positions, in other words, an increased variety of physical activities.

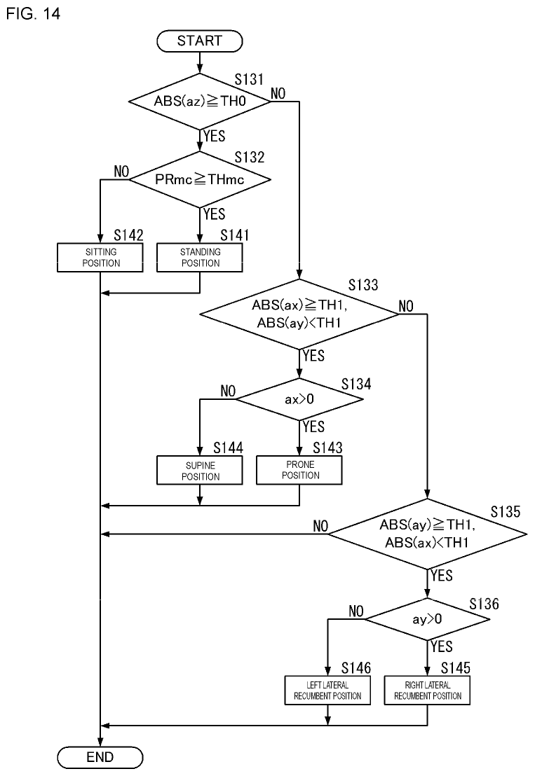

This kind of body position detection can be realized by performing, for example, a process following a flowchart illustrated in FIG. 14. In particular, FIG. 14 is a flowchart illustrating a third example of the physical activity monitoring method according to the first exemplary embodiment.

When the z-axis acceleration index absolute value ABS(az) is equal to or larger than the threshold TH0 (YES in S131), and the muscle activity index PRmc is equal to or larger than the threshold THmc (YES in S132), the computation unit 40 detects the standing position (S141). When the z-axis acceleration index absolute value ABS(az) is equal to or larger than the threshold TH0+ (YES in S131), and the muscle activity index PRmc is smaller than the threshold THmc (NO in S132), the computation unit 40 detects the sitting position (e.g., chair sitting position) (S142).

When the z-axis acceleration index absolute value ABS(az) is smaller than the threshold TH0 (NO in S131), and additionally, when the x-axis acceleration index absolute value ABS(ax) is equal to or larger than the threshold TH1, and the y-axis acceleration index absolute value ABS(ay) is smaller than the threshold TH1 (YES in S133), the computation unit 40 moves to an operation of detecting the lying position (prone position) or the lying position (e.g., supine position). When the x-axis acceleration index ax is a plus value (positive value) (YES in S134), the computation unit 40 detects the lying position (e.g., prone position) (S143). When the x-axis acceleration index ax is a minus value (negative value) (NO in S134), the computation unit 40 detects the lying position (e.g., supine position) (S144).

When the x-axis acceleration index absolute value ABS(ax) is equal to or larger than the threshold TH1, and the y-axis acceleration index absolute value ABS(ay) is not smaller than the threshold TH1 (NO in S133), and additionally, when the y-axis acceleration index absolute value ABS(ay) is equal to or larger than the threshold TH1, and the x-axis acceleration index absolute value ABS(ax) is smaller than the threshold TH1 (YES in S135), the computation unit 40 moves to an operation of detecting the lying position (e.g., right lateral recumbent position) or the lying position (e.g., left lateral recumbent position). When the y-axis acceleration index ay is a plus value (positive value) (YES in S136), the computation unit 40 detects the lying position (e.g., right lateral recumbent position) (S145). When the y-axis acceleration index ay is a minus value (negative value) (NO in S136), the computation unit 40 detects the lying position (e.g., left lateral recumbent position) (S146).

(Second Exemplary Embodiment)

A physical activity monitoring device according to a second exemplary embodiment will be described with reference to the drawings. The physical activity monitoring device according to the second embodiment differs from the physical activity monitoring device according to the first embodiment in that the first monitoring signal and the second monitoring signal obtained by the muscle activity sensor and the acceleration sensors attached to each of two legs are used to detect physical activities (for example, multiple kinds of body positions). Other configurations of the physical activity monitoring device according to the second embodiment are the same as the physical activity monitoring device according to the first embodiment, descriptions thereof are omitted.

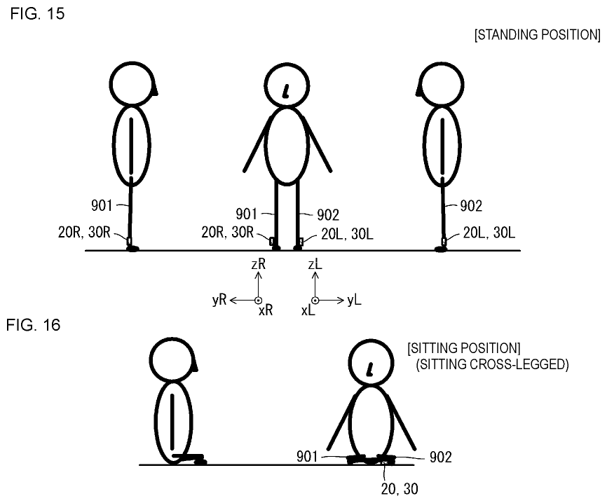

FIG. 15 is a simplified illustration depicting a manner of attachment of the physical activity monitoring device according to the second exemplary embodiment. As illustrated in FIG. 15, the physical activity monitoring device according to the second embodiment includes muscle activity sensors 20R and 20L and acceleration sensors 30R and 30L.

The muscle activity sensor 20R and the acceleration sensor 30R are attached close to the ankle 91 of the right leg 901. Similarly, the muscle activity sensor 20L and the acceleration sensor 30L are attached close to the ankle 91 of the left leg 902.

An x-axis direction xR of the acceleration sensor 30R is identical to an x-axis direction xL of the acceleration sensor 30L. A z-axis direction zR of the acceleration sensor 30R is identical to a z-axis direction zL of the acceleration sensor 30L.

A y-axis direction yR of the acceleration sensor 30R is opposite to a y-axis direction yL of the acceleration sensor 30L. More specifically, the plus direction of the y-axis direction yR of the acceleration sensor 30R directs away from the left leg 902 with respect to the right leg 901. The plus direction of the y-axis direction yL of the acceleration sensor 30L directs away from the right leg 901 with respect to the left leg 902.

In the standing position illustrated in FIG. 15, the z-axis acceleration index azR of the acceleration sensor 30R and the z-axis acceleration index azL of the acceleration sensor 30L are relatively large minus values, whereas the x-axis acceleration index axR and the y-axis acceleration index ayR of the acceleration sensor 30R and the x-axis acceleration index axL and the y-axis acceleration index ayL of the acceleration sensor 30L are equal to a reference value (for example, 0).

FIG. 16 is a simplified illustration depicting a cross-legged sitting position in the state in which the physical activity monitoring device according to the second embodiment is attached. In the cross-legged sitting position illustrated in FIG. 16, the outside of the right leg 901 and the outside of the left leg 902 both face downwards in the vertical direction. As a result, the y-axis acceleration index ayR and the y-axis acceleration index ayL are both relatively large plus values.

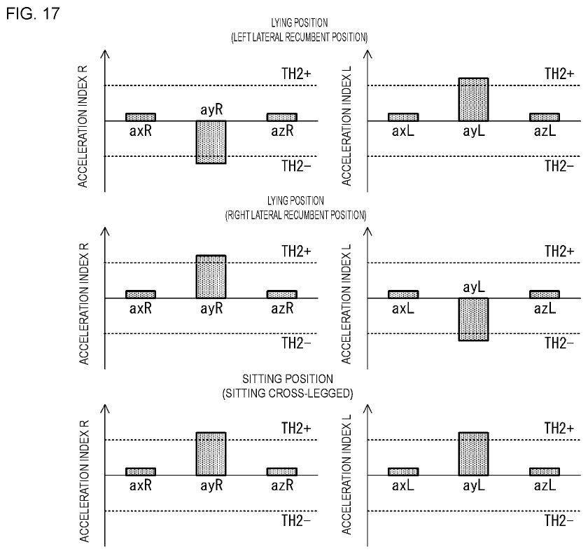

FIG. 17 provides graphs illustrating an example of values of the acceleration index of the lying position (e.g., left lateral recumbent position), the lying position (e.g., right lateral recumbent position), and the sitting position (e.g., a cross-legged sitting position). In FIG. 17, the reference value of acceleration (value of no acceleration) is 0, for example.

As illustrated in FIG. 17, in the lying position (e.g., left lateral recumbent position), the y-axis acceleration index ayR is a relatively large minus value (negative value), whereas the y-axis acceleration index ayL is a relatively large plus value (positive value). In the lying position (e.g., right lateral recumbent position), the y-axis acceleration index ayR is a relatively large plus value (positive value), whereas the y-axis acceleration index ayL is a relatively large minus value (negative value). In the sitting position (e.g., cross-legged sitting position), the y-axis acceleration index ayR and the y-axis acceleration index ayL are both relatively large plus values (positive values).

By using these patterns of the y-axis acceleration index, the computation unit 40 can be configured to detect the sitting position (e.g., cross-legged sitting position) in addition to the multiple kinds of body positions described above in the first embodiment.

Because the muscle activity sensor 20R is attached to the right leg 901, and the muscle activity sensor 20L of the left leg 902 is attached, the computation unit 40 can also be configured to detect a two-leg standing position, a right-leg standing position, and a left-leg standing position.

Specifically, in the two-leg standing position, muscles and tendons of both legs are active to a large extent, the muscle activity index PRmcR of the muscle activity sensor 20R and the muscle activity index PRmcL of the muscle activity sensor 20L are both equal to or larger than the threshold THmc.

In the right-leg standing position, muscles and tendons of the right leg 901 are active to a large extent, whereas muscles and tendons of the left leg 902 are almost inactive. Thus, the muscle activity index PRmcR of the muscle activity sensor 20R is equal to or larger than the threshold THmc, and the muscle activity index PRmcL of the muscle activity sensor 20L is smaller than the threshold THmc.

In the left-leg standing position, muscles and tendons of the left leg 902 are active to a large extent, whereas muscles and tendons of the right leg 901 are almost inactive. Thus, the muscle activity index PRmcL of the muscle activity sensor 20L is equal to or larger than the threshold THmc, and the muscle activity index PRmcR of the muscle activity sensor 20R is smaller than the threshold THmc.

By using these results, the computation unit 40 can be configured to detect the two-leg standing position, the right-leg standing position, and the left-leg standing position in an individual manner.

(Specific Example of Detection of Body Position by Computation Unit 40)

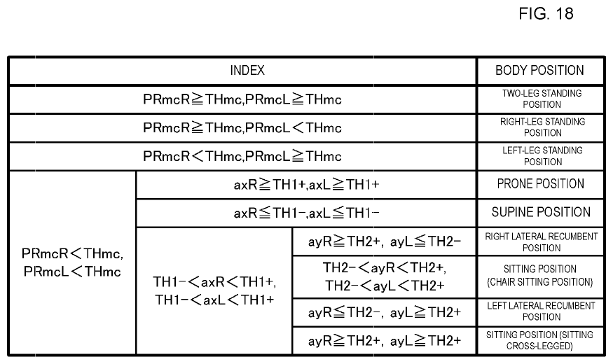

FIG. 18 is a fourth table indicating the relationship between the muscle activity index and the acceleration index, and the body position. The example in FIG. 18 indicates the case without using the z-axis acceleration index az for body position detection, but it is reiterated that the z-axis acceleration index az may also be used to detect the body position as described above in the first embodiment.

After calculating the muscle activity index PRmcR, the muscle activity index PRmcL, the x-axis acceleration index axR, the x-axis acceleration index axL, the y-axis acceleration index ayR, and the y-axis acceleration index ayL, the computation unit 40 can be configured to detect a body position in accordance with rules indicated in FIG. 18 by using these indexes.

Specifically, when the muscle activity index PRmcR and the muscle activity index PRmcL are equal to or larger than the threshold THmc, the computation unit 40 can be configured to detect the two-leg standing position. When the muscle activity index PRmcR is equal to or larger than the threshold THmc, and the muscle activity index PRmcL is smaller than the threshold THmc, the computation unit 40 detects the right-leg standing position. When the muscle activity index PRmcL is equal to or larger than the threshold THmc, and the muscle activity index PRmcR is smaller than the threshold THmc, the computation unit 40 detects the left-leg standing position.

When the muscle activity index PRmcR and the muscle activity index PRmcL are smaller than the threshold THmc, the computation unit 40 detects a body position as described below in accordance with the x-axis acceleration index axR, the x-axis acceleration index axL, the y-axis acceleration index ayR, and the y-axis acceleration index ayL.

When the x-axis acceleration index axR and the x-axis acceleration index axL are equal to or larger than the threshold TH1+, the computation unit 40 detects the lying position (e.g., prone position).

When the x-axis acceleration index axR and the x-axis acceleration index axL are equal to or smaller than the threshold TH1−, the computation unit 40 detects the lying position (e.g., supine position).

When the x-axis acceleration index axR and the x-axis acceleration index axL are larger than the threshold TH1− and smaller than the threshold TH1+, the computation unit 40 detects a body position as described below in accordance with the y-axis acceleration index ayR and the y-axis acceleration index ayL.

When the y-axis acceleration index ayR is equal to or larger than the threshold TH2+, and the y-axis acceleration index ayL is equal to or smaller than the threshold TH2−, the computation unit 40 detects the lying position (e.g., right lateral recumbent position). When the y-axis acceleration index ayR is equal to or smaller than the threshold TH2−, and the y-axis acceleration index ayL is equal to or larger than the threshold TH2+, the computation unit 40 detects the lying position (e.g., left lateral recumbent position).

When the y-axis acceleration index ayR and the y-axis acceleration index ayL are equal to or larger than the threshold TH2+, the computation unit 40 detects the sitting position (e.g., cross-legged sitting position). When the y-axis acceleration index ayR and the y-axis acceleration index ayL are larger than the threshold TH2− and smaller than the threshold TH2+, the computation unit 40 detects the sitting position (e.g., chair sitting position).

As described above, with the use of the configurations and operations of the present embodiment, the physical activity monitoring device 10 can be configured to perform detection of multiple kinds of body positions including differentiation between the two-leg standing position and the single-leg standing positions, and detection of the cross-legged sitting position, in other words, detection of an increased variety of physical activities.

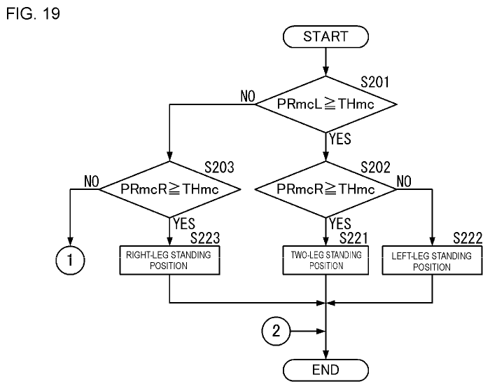

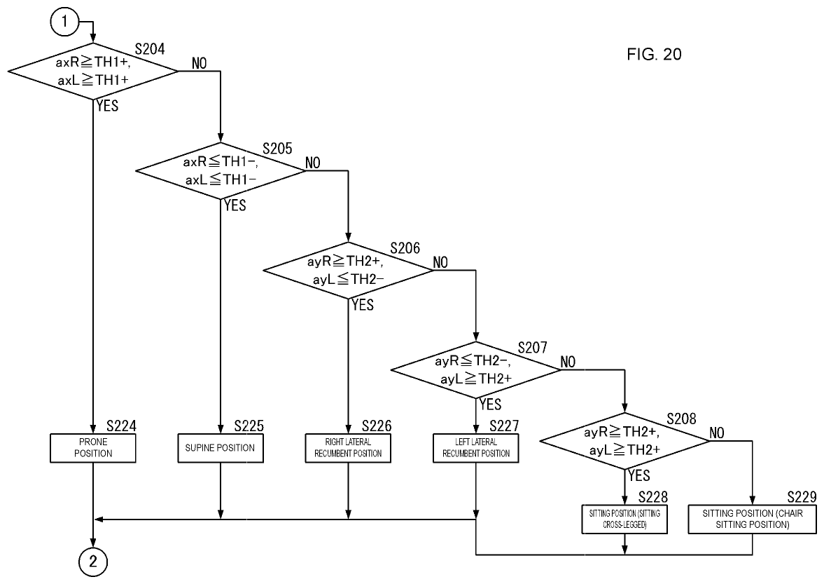

This kind of body position detection can be realized by performing, for example, a process following a flowchart illustrated in FIGS. 19 and 20. In particular, FIGS. 19 and 20 provides a flowchart illustrating a first example of the physical activity monitoring method according to the second exemplary embodiment.

When the muscle activity index PRmcL is equal to or larger than the threshold THmc (YES in S201), and the muscle activity index PRmcR is equal to or larger than the threshold THmc (YES in S202), the computation unit 40 detects the two-leg standing position (S221).

When the muscle activity index PRmcL is equal to or larger than the threshold THmc (YES in S201), and the muscle activity index PRmcR is smaller than the threshold THmc (NO in S202), the computation unit 40 detects the left-leg standing position (S222).

When the muscle activity index PRmcL is smaller than the threshold THmc (NO in S201), and the muscle activity index PRmcR is equal to or larger than the threshold THmc (YES in S203), the computation unit 40 detects the right-leg standing position (S223).

When the muscle activity index PRmcL is smaller than the threshold THmc (NO in S201), and the muscle activity index PRmcR is smaller than the threshold THmc (NO in S203), the computation unit 40 proceeds to step S204 (proceeds from FIG. 19 to FIG. 20).

When the x-axis acceleration index axR is equal to or larger than the threshold TH1+, and the x-axis acceleration index axL is equal to or larger than the threshold TH1+ (YES in S204), the computation unit 40 detects the lying position (e.g., prone position) (S224).

When the x-axis acceleration index axR is not equal to or larger than the threshold TH1+, and the x-axis acceleration index axL is not equal to or larger than the threshold TH1+ (NO in S204), and additionally, when the x-axis acceleration index axR is equal to or smaller than the threshold TH1−, and the x-axis acceleration index axL is equal to or smaller than the threshold TH1− (YES in S205), the computation unit 40 detects the lying position (e.g., supine position) (S225).

When the x-axis acceleration index axR is not equal to or smaller than the threshold TH1−, and the x-axis acceleration index axL is not equal to or smaller than the threshold TH1− (NO in S205), and additionally, when the y-axis acceleration index ayR is equal to or larger than the threshold TH2+, and the y-axis acceleration index ayL is equal to or smaller than the threshold TH2− (YES in S206), the computation unit 40 detects the lying position (e.g., right lateral recumbent position) (S226).

When the y-axis acceleration index ayR is not equal to or larger than the threshold TH2+, and the y-axis acceleration index ayL is not equal to or smaller than the threshold TH2− (NO in S206), and additionally, when the y-axis acceleration index ayR is equal to or smaller than the threshold TH2−, and the y-axis acceleration index ayL is equal to or larger than the threshold TH2+ (YES in S207), the computation unit 40 detects the lying position (e.g., left lateral recumbent position) (S227).

When the y-axis acceleration index ayR is not equal to or smaller than the threshold TH2−, and the y-axis acceleration index axL is not equal to or larger than the threshold TH2+ (NO in S207), and additionally, when the y-axis acceleration index ayR and the y-axis acceleration index axL are equal to or larger than the threshold TH2+ (YES in S208), the computation unit 40 detects the sitting position (e.g., cross-legged sitting position) (S228); otherwise (NO in S206), the computation unit 40 detects the sitting position (e.g., chair sitting position) (S229).

Similarly to the first embodiment, body position detection using the z-axis accelerations azR and azL and body position detection using the absolute value of acceleration can be applied to the second exemplary embodiment.

(Derivative Example of Functional Configuration)

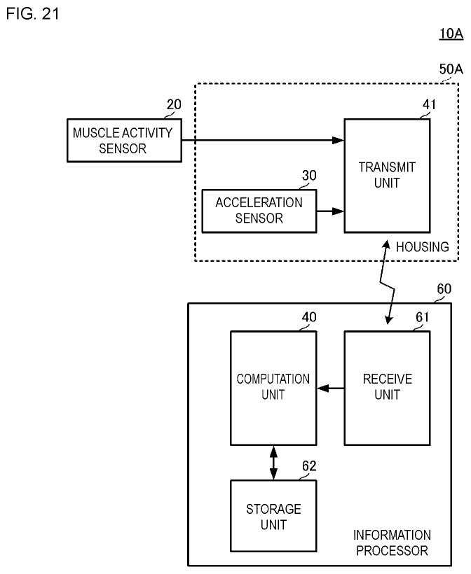

The above description has explained the configuration in which all the functional units are arranged in, for example, the body supporter 500. However, at least the muscle activity sensor 20 and the acceleration sensor 30 need to be arranged in the body supporter 500. For example, as illustrated in FIG. 21, the computation unit 40 can be disposed apart from the body supporter 500. FIG. 21 is a functional block diagram of a physical activity monitoring device according to an additional exemplary embodiment.

As illustrated in FIG. 21, the physical activity monitoring device 10A includes the muscle activity sensor 20, the acceleration sensor 30, a transmit unit 41 (e.g., a transmitted), and an information processor 60. The information processor 60 includes the computation unit 40, a receive unit 61 (e.g., a receiver), and a storage unit 62.

The transmit unit 41 can be implemented by, for example, an electronic circuit, and be configured to transmit the second monitoring signal from the muscle activity sensor 20 and the first monitoring signal from the acceleration sensor 30 to the receive unit 61 of the information processor 60. The transmit unit 41 is housed in, for example, the housing 50A together with the acceleration sensor 30.

According to an exemplary aspect, the information processor 60 can be implemented by, for example, a known personal computer or an information communication terminal. The receive unit 61 receives the first monitoring signal and the second monitoring signal from the transmit unit 41 and outputs the first monitoring signal and the second monitoring signal to the computation unit 40.

The computation unit 40 can be configured to perform physical activity monitoring including body position detection as described above by using the first monitoring signal and the second monitoring signal. After obtaining the first monitoring signal and the second monitoring signal, the computation unit 40 stores the first monitoring signal and the second monitoring signal in the storage unit 62. As a result, the computation unit 40 can perform physical activity monitoring including body position detection in, for example, an offline manner. The computation unit 40 can store results of physical activity monitoring in the storage unit 62. The computation unit 40 can additionally display the results of physical activity monitoring on a display unit such as a liquid crystal display, which is not illustrated in the drawing.

In general, it is noted that the above description has explained the configuration in which a piezoelectric sensor is used as the muscle activity sensor. In general, when the piezoelectric sensor is used according to the exemplary embodiment, a signal caused by a tremor can be detected as a signal representing a muscle activity. For purposes of this disclosure, the term “tremor” is used to denote, for example, involuntary motion indicating rhythmic muscle activities. For example, a tremor according to an exemplary aspect can be a small and rapid postural tremor that occurs in ordinary people. This kind of postural tremor is referred to as physiological tremor, and the frequency of this kind of postural tremor ranges, for example, from 8 to 12 Hz. It is also noted that shaking that occurs in patients including Parkinsonian patients is pathologic tremor, and the frequency of this kind of tremor ranges, for example, from 4 to 7 Hz, which is not considered as a tremor detected by the muscle activity sensor as described herein according to an exemplary aspect.

Using a tremor as the detection signal provides advantageous aspects when compared with using myoelectric signals. For example, it is possible to detect (e.g., measure) a tremor without direct attachment to a surface (for example, skin) of a detection target object, such as a human body, for example. By detecting tremor, expansion and contraction of muscles can be detected. By detecting tremor, changes due to muscle fatigue can be detected.

Moreover, it is noted that the muscle activity sensor is not limited to a piezoelectric sensor, but may be, for example, an acceleration sensor or a microphone. The muscle activity sensor may be another kind of sensor capable of detecting signals of, for example, about 10 Hz.

It is also generally noted that the configurations and operations of the embodiments can be combined with each other as appropriate, and it is possible to achieve effects and advantages corresponding to individual combinations thereof.

### REFERENCE SIGNS LIST

20, 20L, 20R muscle activity sensor
30, 30L, 30R acceleration sensor
40 computation unit
41 transmit unit
50, 50A housing
60 information processor
61 receive unit
62 storage unit
500 body supporter

1. A physical activity monitoring device comprising:
an acceleration sensor configured to attach to a leg of a user and to output a first monitoring signal corresponding to an activity of the leg;
a muscle activity sensor configured to attach to the leg and to output a second monitoring signal corresponding to an activity of at least one of a muscle and a tendon of the leg; and
a computation unit configured to detect a load condition of a body of the user by using the first monitoring signal and the second monitoring signal, with the load condition indicating a body position of the body of the user.

2. The physical activity monitoring device according to claim 1, wherein the computation unit is configured to detect the load condition by using a level of the first monitoring signal and a level of the second monitoring signal.

3. The physical activity monitoring device according to claim 2, wherein the computation unit is configured to detect the load condition based on an integration time of the level of the first monitoring signal and an integration time of the level of the second monitoring signal.

4. The physical activity monitoring device according to claim 1, wherein the acceleration sensor is configured to detect an acceleration in a direction connecting a toe tip and a heel of the leg of the user.

5. The physical activity monitoring device according to claim 1, wherein the acceleration sensor is configured to detect an acceleration in a direction perpendicular to left and right sides of the leg of the user.

6. The physical activity monitoring device according to claim 1, wherein the muscle activity sensor includes a piezoelectric sensor configured to output the second monitoring signal in accordance with a tremor of the leg of the user.

7. The physical activity monitoring device according to claim 1, wherein the muscle activity sensor includes an electromyography sensor configured to output the second monitoring signal based on a muscle activity of the leg of the user.

8. The physical activity monitoring device according to claim 1, wherein the computation unit is configured to differentiate at least two kinds of body positions of the wearer user as the load condition of the body, with the at least two kinds of body positions selected from the group of a lying position, a sitting position, and a standing position.

9. The physical activity monitoring device according to claim 8,
wherein the acceleration sensor is configured to detect an acceleration in a direction connecting a toe tip and a heel of the leg, and
wherein the computation unit is configured to differentiate, based on the first monitoring signal that represents information including the acceleration in the direction connecting the toe tip and the heel of the leg, between the lying position, and the sitting position and the standing position.

10. The physical activity monitoring device according to claim 1, wherein the computation unit is configured to differentiate, based on the second monitoring signal, between a lying position or a sitting position, and a standing position of the body position of the user.

11. The physical activity monitoring device according to claim 10, wherein the computation unit is configured to differentiate, based on the first monitoring signal, between the lying position and the sitting position.

12. The physical activity monitoring device according to claim 11, wherein the computation unit is configured to differentiate, based on a sign of the first monitoring signal, between a supine position and a prone position in the lying position.

13. The physical activity monitoring device according to claim 11, wherein the computation unit is configured to detect a lateral recumbent position in the lying position based on the first monitoring signal.

14. The physical activity monitoring device according to claim 13, wherein the computation unit is configured to differentiate, based on a sign of the first monitoring signal, between a left lateral recumbent position and a right lateral recumbent position in the lateral recumbent position.

15. The physical activity monitoring device according to claim 11, wherein the acceleration sensor is configured to attach to two legs, which includes the leg, of the user.

16. The physical activity monitoring device according to claim 15, wherein the computation unit is configured to detect a cross-legged sitting position in the sitting position based on the first monitoring signal outputted by the acceleration sensor.

17. The physical activity monitoring device according to claim 1, wherein the muscle activity sensor is configured to attach to two legs, which includes the leg, of the user.

18. The physical activity monitoring device according to claim 17, wherein the computation unit is configured to differentiate, based on the second monitoring signal outputted by the muscle activity sensor attached to the two legs, between a two-leg standing position and a single-leg standing position.

19. The physical activity monitoring device according to claim 1, wherein the computation unit comprises a microcomputer configured to calculate the body position of the body of the user based on the first monitoring signal and the second monitoring signal.

20. The physical activity monitoring device according to claim 1, further comprising a housing with the acceleration sensor, the muscle activity sensor and the computation unit disposed therein, with the housing being constructed to position the muscle activity sensor around an ankle circumference of the leg of the user when the housing is attached to the leg of the user.
